(19) United States
(12) Patent Application Publication
(10) Pub. No.: US 2004/0144388 A1
(43) Pub. Date: Jul. 29, 2004

(54) Restraint device

(76) Inventors: Micheal James Wingrave Hall, Stansted Essex (GB); Andrew Paul Bareham, Essex (GB)

Correspondence Address: Christopher J Fildes, Fildes & Outland, Suite 2, 20916 Mack Avenue, Grosse Pointe Woods, MI, 48236, US

(21) Appl. No.: 10/482,908

(22) PCT Filed: Jul. 4, 2002
(86) PCT No.: PCT/GB02/03081

(30) Foreign Application Priority Data
Jul. 4, 2001 (GB) 0116294.0

(51) Int. Cl. A61F 5/37

(52) U.S. Cl. 128/846

(57) ABSTRACT

A restraint device (1) comprises a belt (2) and complementary quick-fastening means (3, 4) which are associated with the belt (2) and which, when quick-fastened together, render the belt (2) into a secure closed loop (11) of desired length. The device (1) may be applied to the arms and torso of a person to be restrained or the wrists or ankles thereof.

### DESCRIPTION

This invention relates to a restraint device and is especially, but not exclusively, related to such a device for restraining human beings and, in certain circumstances, certain types of animal.

Restraint devices are used by numerous agencies for the restraint and control of violent or potentially violent persons. Known types of such devices can cause discomfort to persons to which they are applied and, in certain circumstances, can cause injury if applied incorrectly or if the person is larger than average. Also, known restraint devices necessitate a detailed training programme for the user. Further, such devices can be complicated to apply and may necessitate the user being in close proximity to a person to be restrained. This can leave the user vulnerable during what can be a protracted operation in a time of conflict and confrontation. Further, known restraint devices do not always fully restrain the arms and/or legs of a person being restrained and may be incapable of fully confining or restraining a person in a seat or chair, if necessary.

An object of the invention is to provide a restraint device which overcomes, or at least substantially reduces, the disadvantages associated with known devices and which is simple, quick and safe to apply to a person to be restrained.

Accordingly, the invention provides a restraint device comprising a belt and complementary quick-fastening means which are associated with the belt and which, when quick-fastened together, render the belt into a secure, closed loop of desired length.

Preferably, the complementary quick-fastening means also constitute quick-release means.

Also, the, belt is preferably made of a strong material, such as nylon webbing, leather, plastic strip or any combination thereof.

The complementary, quick-fastening means may be of the loop and hook variety, such as that sold under the Registered Trade Mark “Velcro”, in which case, respective complementary loop and hook portions, in appropriate lengths, can be secured to at least one side of the belt.

In one embodiment of restraint device to be described in more detailed hereinbelow, one end of the belt may be provided with a strong ring or eye, preferably made of a metallic material, with the other end of the belt being threadable through the ring or eye, so that when the device is placed over or otherwise applied to a person to be restrained, the belt can be rapidly adjusted to the required length at which stage the complementary quick-fastening means can be about the restrained person.

If the person to be restrained is sitting in a backed seat, the belt can be so-formed into a secure loop about the torso, arms and seat back, with the complementary quick-fastening means secured together, thereby restraining the person securely within the seat.

The restraint device of the invention enables a user to apply the device to a person to be restrained from behind that person, with a minimum of effort.

In other embodiments, the restraint device can be applied to the wrists and/or ankles of a person to be restrained, thereby securing at least one pair of limbs of the person.

The device is such that it requires a minimum of training for a user and is flexible in its application for restraining the arms, legs or both of a person to be restrained, whilst the user can maintain a safe distance between the person being restrained and himself.

The invention also provides a method of restraining a person, comprising placing around part of that person a belt, including complementary quick-fastening means, to form a secure loop therearound, and securing together the complementary quick-fastening means.

The belt may be adjusted to length before the complementary quick-fastening means are secured together.

In such a method, one end of the belt may be threaded through a strong ring or eye at one end thereof, so that a required length of the belt may be pulled through the ring or eye, to tighten the belt about, say, the torso and arms of a person to be restrained.

Alternatively, a belt with or without length adjustment may be used to secure, say, the wrists and/or ankles of a person to be restrained.

In order that the invention may be more fully understood, preferred embodiments of restraint device in accordance therewith, will now be described by way of example and with reference to the accompanying drawings in which:

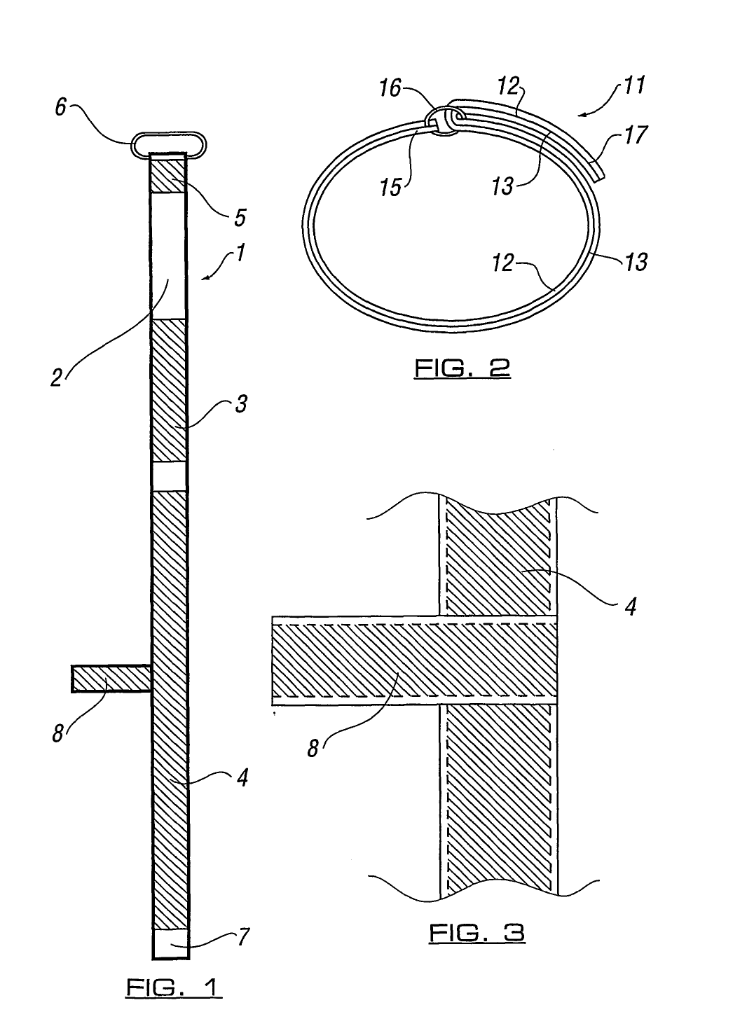

FIG. 1 is a plan view of a first embodiment of restraint device in its open condition;

FIG. 2 is a side view of another embodiment of restraint device shown in its closed loop condition;

FIG. 3 is, on an enlarged scale, a partial view of the restraint device shown in FIG. 1;

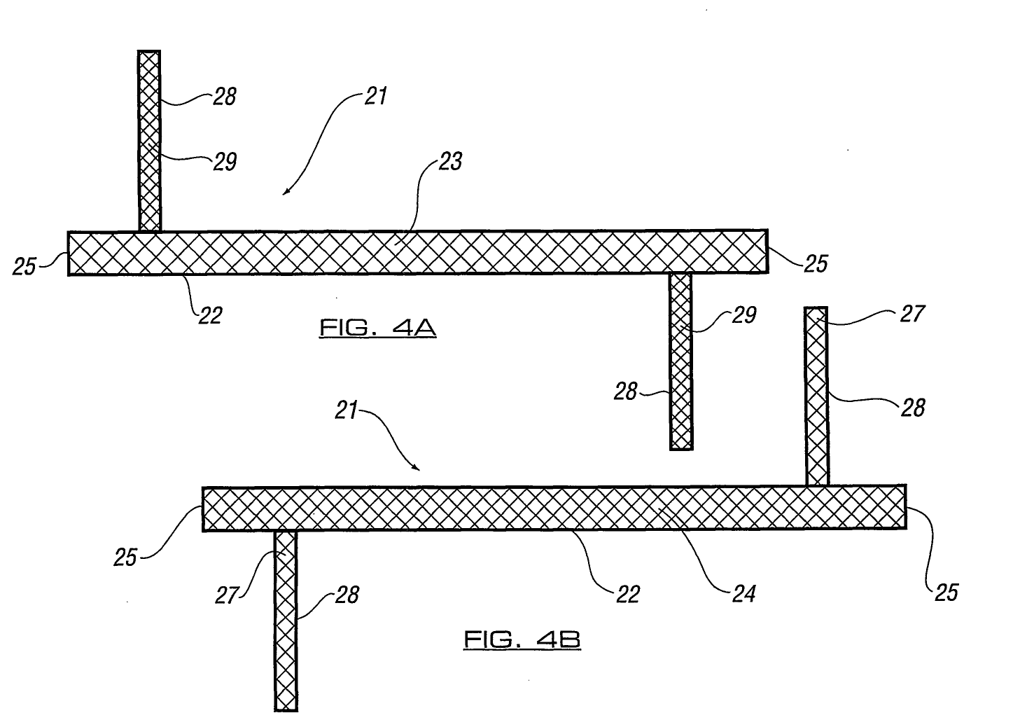

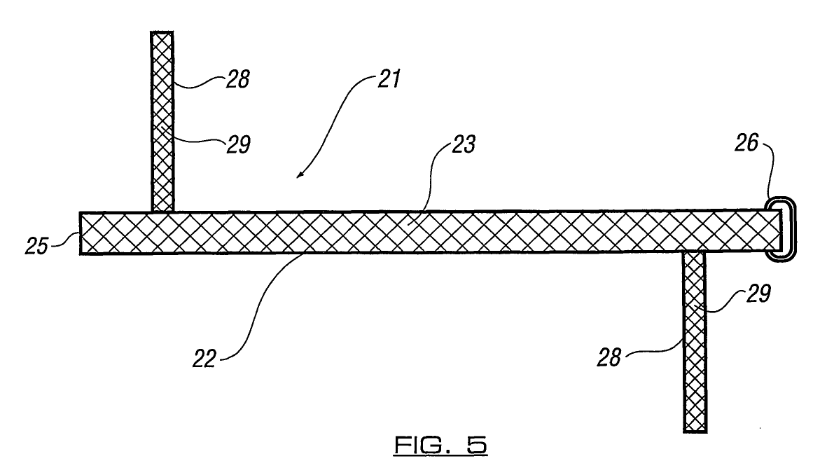

FIGS. 4A and 4B are plan views of respective opposed sides of a further embodiment of restraint device; and

FIG. 5 is a plan view of a modified form of the embodiment shown in FIGS. 4A and 4B.

Referring firstly to FIG. 1 of the accompanying drawings, a first embodiment of restraint device, as indicated generally at 1, comprises a flexible belt 2 made from strong nylon webbing.

Associated with the belt 2 are complementary quick-fastening means in the form of a Velcro fastener with the respective loop and hook portions 3, 4 being secured firmly to one side of the belt 2.

At one end 5 of the belt 2, there is provided a strong metal eye 6 through which the other end 7 of the belt 2 can be threaded.

As also shown in FIG. 3, a locking tab 8 is also secured to the belt 2 at right angles thereto.

The locking tab 8, which may comprise a loop and hook, quick-fastening means, such as respective pieces of Velcro or suitable metallic or plastics locking means, can be folded about two thicknesses of the belt 2 when the belt 2 is formed into a secure, closed loop of desired length about a person to be restrained.

Such a secure, closed loop is shown generally at 11 in FIG. 2 but, in this case, the belt 12 is provided with Velcro quick-fastening means 13 along substantially the whole of its length.

As can be seen from FIG. 2, one end 17 of the belt 12 has been threaded through a strong metal eye 16 provided at the other end 15 of the belt 12, with the belt having been pulled through the eye 16, such that the so-formed, secure closed loop 11 is of the desired length to restrain a person. The locking tab 8 can be used with the second embodiment of restraint device 11 shown in FIG. 2, even though it is not actually shown in that Figure.

Thus, it can be seen that the restraint device 1, 11, as described above in relation to FIGS. 1 to 3, can be used by passing the end 7, 17 of the belt 2, 12 through the eye 6, 16, to form a lasso-type loop 11 which can be passed over the head and shoulders of a person to be restrained to such a degree that it pins the upper arms of that person to his or her chest or other part of the torso.

The belt 2, 12 is then pulled through the eye 6, 16, thereby tightening the restraint device about the person until that person is restrained and is then folded about the eye 6, 16, so that the quick-fastening portions 3, 4 are engaged together, to provide the secure, closed loop 11 of desired length.

The locking tab 8 can then be wrapped around or applied through the belt 2, 12 and fastened, thereby reinforcing the quick-fastening Velcro fasteners 3, 4 on the main, central portion of the belt 2, 12.

The restraint device can be used to restrain a violent person on board, say, an aircraft, by passing the untightened loop 11 over the head and shoulders of the person, as well as the back of the seat in which that person is sitting, and then tightened and retained as a secure, closed loop of desired length. Thus, the arms of the person to be restrained would be pinned to the side of his or her torso, thereby preventing him or her from striking any other person and confining him or her to his or her seat.

Referring now to FIGS. 4A and 4B of the accompanying drawings, a further embodiment of restraint device, as indicated generally at 21, comprises a flexible belt 22 made from, say, strong nylon webbing.

Associated with the belt 22 are complementary quick-fastening means in the form of a Velcro fastener with the respective loop and hook portions 23, 24 being secured firmly to respective sides of the belt 22 and extending along the entire lengths thereof.

Adjacent opposed ends 25 of the belt 22 respective locking tabs 28 are secured firmly to the belt 22 at right angles thereto.

Each locking tab 28 comprises a loop and hook, quick-fastening means, such as respective pieces 27, 29 of Velcro on respective opposed sides of the tab 28, extending along the entire lengths thereof.

The restraint device 21 can be wound or wrapped readily around the wrists and/or ankles of a person to be restrained.

The quick-fastening portions 23, 24 then engage together, to provide a secure, closed loop around the wrists and/or ankles of the so-restrained person.

At least one of the locking tabs 28 is then employed to fasten yet further the belt 22 around the wrists and/or ankles of the restrained person and fastened, via the complementary loop and hook, quick-fastening means 27, 29 of its own and/or in combination with those 23, 24 of the belt 22.

The complementary quick-fastening means may, as discussed above, also be of a quick-release nature, in which case, the restraint device can be loosened from a restrained person quite readily at the appropriate time.

In a modified form of the restraint device 21 described above with reference to FIGS. 4A and 4B, a strong eye 26, such as the eye 16 of the restraint device 1, 11 described above in relation to FIGS. 1 to 3, is provided at one end of the belt 22, for receiving therethrough the other end 25 of the belt 22.

1. A restraint device comprising a belt and complementary quick-fastening means which are associated with the belt and which, when quick-fastened together, render the belt into a secure closed loop of desired length.

2. A device according to claim 1, wherein the complementary quick-fastening means also constitute quick-release means.

3. A device according to claim 1 or 2, wherein the belt is made of a strong material, such as nylon webbing, leather, plastic strip or any combination thereof.

4. A device according to claim 1, 2 or 3, wherein the complementary, quick-fastening means is of the loop and hook variety.

5. A device according to claim 4, wherein loop and hook portions, in appropriate lengths, are secured to at least one side of the belt.

6. A device according to any preceding claim, wherein one end of the belt has a strong ring or eye, preferably made of a metallic material, with the other end of the belt being threadable through the ring or eye, so that when the device is placed over or otherwise applied to a person to be restrained, the belt can be rapidly adjusted to the required length at which stage the complementary quick-fastening means can be secured together, to provide a secure loop about the restrained person.

7. A device according to any preceding claim, wherein the belt can be so-formed into a secure loop about the torso, arms and seat back, with the complementary quick-fastening means secured together, thereby restraining the person securely within the seat.

8. A method of restraining a person, comprising placing around that person, or around the wrists or ankles thereof, a belt, including complementary quick-fastening means, to form a secure loop therearound, adjusting the length of the so-formed loop about the person, or about the wrists or ankles thereof, and securing together the complementary quick-fastening means.

9. A method according to claim 8, wherein one end of the belt is threaded through a strong ring or eye at one end thereof, so that a required length of the belt may be pulled through the ring or eye, to tighten the belt about, say, the torso and arms of a person, or the wrists or ankles thereof, to be restrained.
